(19) United States
(12) Patent Application Publication
(10) Pub. No.: US 2022/0061972 A1
(43) Pub. Date: Mar. 3, 2022

(54) HANDS-FREE DENTAL SUCTION DEVICE

(71) Applicant: Hossein Javid, Inglewood, CA (US)

(72) Inventor: Hossein Javid, Inglewood, CA (US)

(21) Appl. No.: 17/410,458

(22) Filed: Aug. 24, 2021

Related U.S. Application Data
(60) Provisional application No. 63/070,079, filed on Aug. 25, 2020.

(51) Int. Cl. A61C 17/12

(52) U.S. Cl. CPC A61C 17/12

(57) ABSTRACT

An exemplary hands-free dental suction device includes an oral partition element, a suction interface portion, a first suction tube and an aerosol capture shroud. The suction interface portion is placeable in fluid communication with a suction source. The first suction tube may have a partition interface attached to the oral partition element, and may extend in fluid communication between the partition interface and the suction interface portion. The aerosol capture shroud may have an aerosol inlet end and an aerosol discharge end. The aerosol inlet end may be larger than the aerosol discharge end, which may thereby give the aerosol capture shroud a flared shape. The aerosol capture shroud may be affixed to an external surface of the first suction tube. The first suction tube may include an intermediate suction port by which the aerosol discharge end is in fluid communication with the first suction tube.

### RELATED APPLICATIONS

This application claims the benefit of U.S. Provisional Application No. 63/070,079 filed Aug. 25, 2020, the content of which is incorporated by this reference its entirety for all purposes as if fully set forth herein.

### TECHNICAL FIELD

The present disclosure relates generally to the field of dental suction devices.

### SUMMARY

Certain deficiencies of the prior art may be overcome by the provision of a hands-free dental suction device, examples of which are disclosed herein.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1 is a diagrammatic perspective view of a first example dental suction device in accordance with the present disclosure;

FIG. 2 is a further diagrammatic perspective view of the dental suction device shown in FIG. 1;

FIG. 3 is yet a further diagrammatic perspective view of the dental suction device shown in FIG. 1;

FIG. 4 is a diagrammatic side view of the dental suction device shown in FIG. 1;

FIG. 5 is a diagrammatic top view of the dental suction device shown in FIG. 1;

FIG. 6 is a diagrammatic bottom view of the dental suction device shown in FIG. 1;

FIG. 7 is a diagrammatic front view of the dental suction device shown in FIG. 1;

FIG. 8 is a diagrammatic cross-sectional view taken along lines 8-8 in FIG. 7;

FIG. 9 is a diagrammatic top view of the dental suction device shown in FIG. 1;

FIG. 10 is a diagrammatic cross-sectional view taken along lines 9-9 in FIG. 10;

FIG. 11 is a diagrammatic rear view of the dental suction device shown in FIG. 1;

FIG. 12 is a diagrammatic top view of a patient's mouth with the dental suction device of FIG. 1 shown in one example usage, wherein the oral partition element is disposed between the patient's tongue and row of teeth, and the suction interface portion is in fluid communication with a source of suction;

FIG. 13 is a diagrammatic perspective view of a second example dental suction device in accordance with the present disclosure;

FIG. 14 is a further diagrammatic perspective view of the dental suction device shown in FIG. 13;

FIG. 15 is a diagrammatic rear view of the dental suction device shown in FIG. 13;

FIG. 16 is a diagrammatic front view of the dental suction device shown in FIG. 1;

FIG. 17 is a diagrammatic side view of the dental suction device shown in FIG. 13;

FIG. 18 is a diagrammatic top view of the dental suction device shown in FIG. 13;

FIG. 19 is a diagrammatic cross-sectional view taken along lines 19-19 in FIG. 18;

FIG. 20 is a diagrammatic bottom view of the dental suction device shown in FIG. 13;

FIG. 21 is a diagrammatic top view of a patient's mouth with the dental suction device of FIG. 13 shown in one example usage, wherein the oral partition element is disposed outside the patient's row of teeth (inside the adjacent cheek), and the suction interface portion is in fluid communication with a source of suction;

FIG. 22 is a diagrammatic perspective view of a third example dental suction device in accordance with the present disclosure;

FIG. 23 is a further diagrammatic perspective view of the dental suction device shown in FIG. 22;

FIG. 24 is a diagrammatic rear view of the dental suction device shown in FIG. 22;

FIG. 25 is a diagrammatic front view of the dental suction device shown in FIG. 22;

FIG. 26 is a diagrammatic side view of the dental suction device shown in FIG. 22;

FIG. 27 is a diagrammatic top view of the dental suction device shown in FIG. 22;

FIG. 28 is a diagrammatic cross-sectional view taken along lines 28-28 in FIG. 27;

FIG. 29 is a diagrammatic bottom view of the dental suction device shown in FIG. 22;

FIG. 30 is a diagrammatic top view of a patient's mouth with the dental suction device of FIG. 22 shown in one example usage, wherein the oral partition element is disposed outside the patient's row of teeth (inside the adjacent cheek), and the suction interface portion is in fluid communication with a source of suction;

FIG. 31 is a diagrammatic perspective view of a fourth example dental suction device in accordance with the present disclosure;

FIG. 32 is a further diagrammatic perspective view of the dental suction device shown in FIG. 31;

FIG. 33 is a diagrammatic rear view of the dental suction device shown in FIG. 31;

FIG. 34 is a diagrammatic front view of the dental suction device shown in FIG. 31;

FIG. 35 is a diagrammatic side view of the dental suction device shown in FIG. 31;

FIG. 36 is a diagrammatic top view of the dental suction device shown in FIG. 31;

FIG. 37 is a diagrammatic cross-sectional view taken along lines 37-37 in FIG. 36;

FIG. 38 is a diagrammatic bottom view of the dental suction device shown in FIG. 31;

FIG. 39 is a diagrammatic top view of a patient's mouth with the dental suction device of FIG. 31 shown in one example usage, wherein the oral partition element is disposed outside the patient's row of teeth (inside the adjacent cheek), and the suction interface portion is in fluid communication with a source of suction;

FIG. 40 is a diagrammatic perspective view of a fifth example dental suction device in accordance with the present disclosure;

FIG. 41 is a diagrammatic side view of the dental suction device shown in FIG. 40;

FIG. 42 is a diagrammatic perspective view of a sixth example dental suction device in accordance with the present disclosure;

FIG. 43 is a diagrammatic side view of the dental suction device shown in FIG. 42;

FIG. 44 is a diagrammatic perspective view of a seventh example dental suction device without its corresponding oral partition element;

FIG. 45 is a further diagrammatic perspective view of the example dental suction device of FIG. 44;

FIG. 46 is a diagrammatic side view of the example dental suction device of FIG. 44;

FIG. 47 is a diagrammatic front view of the example dental suction device of FIG. 44;

FIG. 48 is a diagrammatic cross-sectional view taken across lines 48-48 in FIG. 47;

FIG. 49 is a diagrammatic top view of the example dental suction device of FIG. 44;

FIG. 50 is a diagrammatic bottom view of the example dental suction device of FIG. 44;

FIG. 51 is a diagrammatic cross-sectional view of the example dental suction device of FIG. 44, but with the corresponding oral partition element;

FIG. 52 is a diagrammatic top view of a patient's mouth with the dental suction device of FIG. 51 shown in one example usage, wherein the oral partition element is disposed outside the patient's row of teeth (inside the adjacent cheek), and the suction interface portion is in fluid communication with a source of suction;

FIG. 53 is a diagrammatic perspective view of an eighth example dental suction device;

FIG. 54 is a diagrammatic side view of the example dental suction device of FIG. 53;

FIG. 55 is a diagrammatic top view of the example dental suction device of FIG. 53;

FIG. 56 is a diagrammatic cross-sectional view taken across lines 56-56 in FIG. 55;

FIG. 57 is a diagrammatic perspective view of a ninth example dental suction device without its corresponding oral partition element;

FIG. 58 is a further diagrammatic perspective view of the example dental suction device of FIG. 57;

FIG. 59 is a diagrammatic side view of the example dental suction device of FIG. 57;

FIG. 60 is a diagrammatic front view of the example dental suction device of FIG. 57; and

FIG. 61 is a diagrammatic cross-sectional view taken across lines 61-61 in FIG. 60.

### DETAILED DESCRIPTION OF THE PREFERRED EMBODIMENTS

Referring now to the drawings, like reference numerals designate identical or corresponding features throughout the several views.

Various example embodiments of a hands-free dental suction device in accordance with the present disclosure are shown generally at 100 in the several drawings presented herewith. A dental suction device 100 may comprise an oral partition element 102, a suction interface portion 104, a first suction tube 108, and an aerosol capture shroud 112. Referring to FIGS. 12, 21, 30, 39, and 52, the suction interface portion 104 may be configured to be in fluid communication with a source of suction 106. The source of suction 106 may be a conventional dental suction unit typically incorporating a vacuum pump, or an equivalent dental suction unit developed at some point in future.

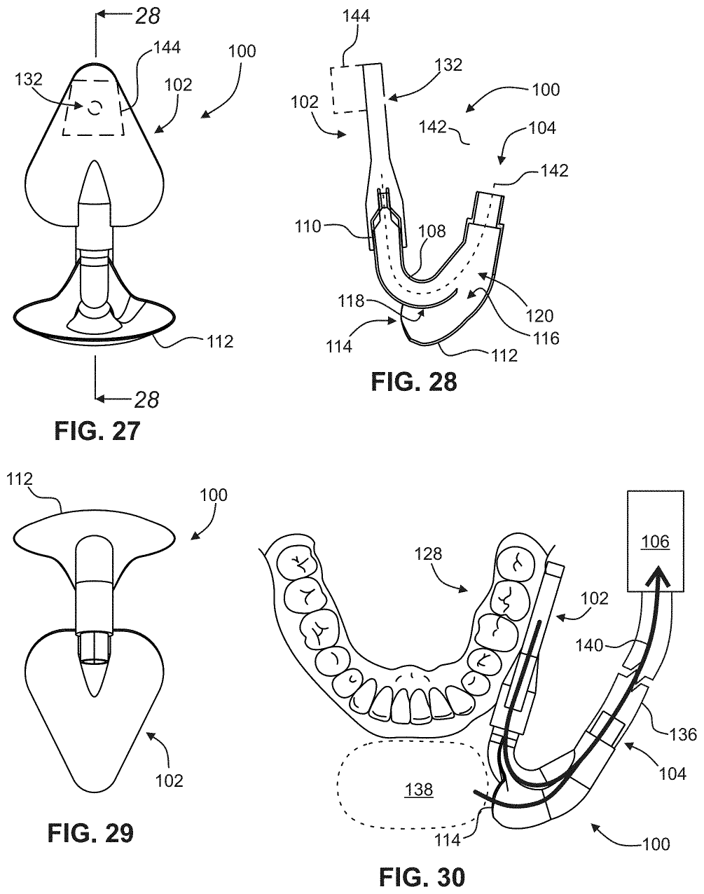

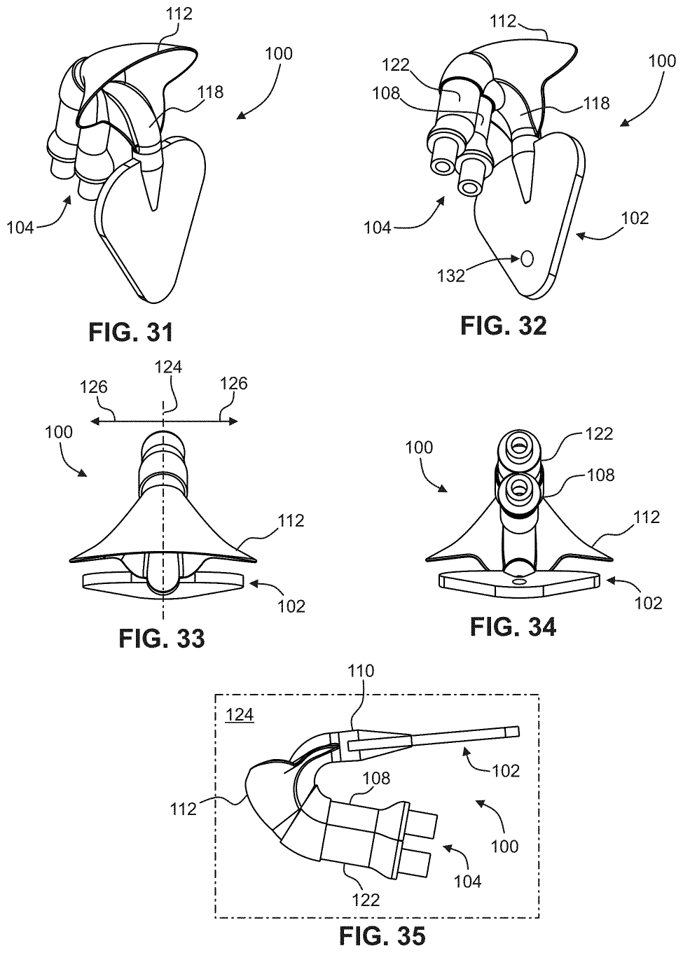

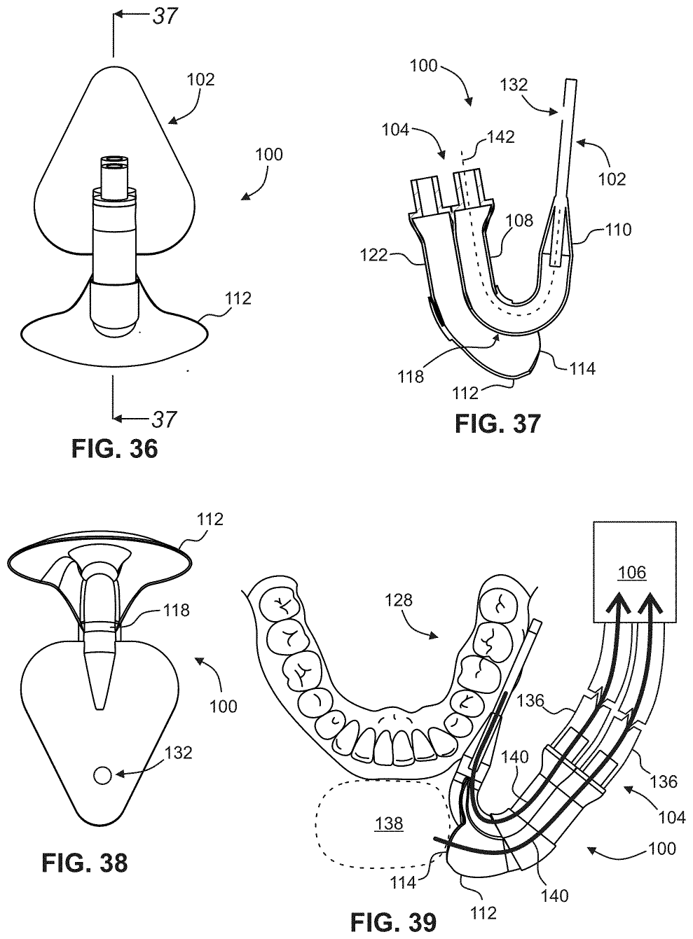

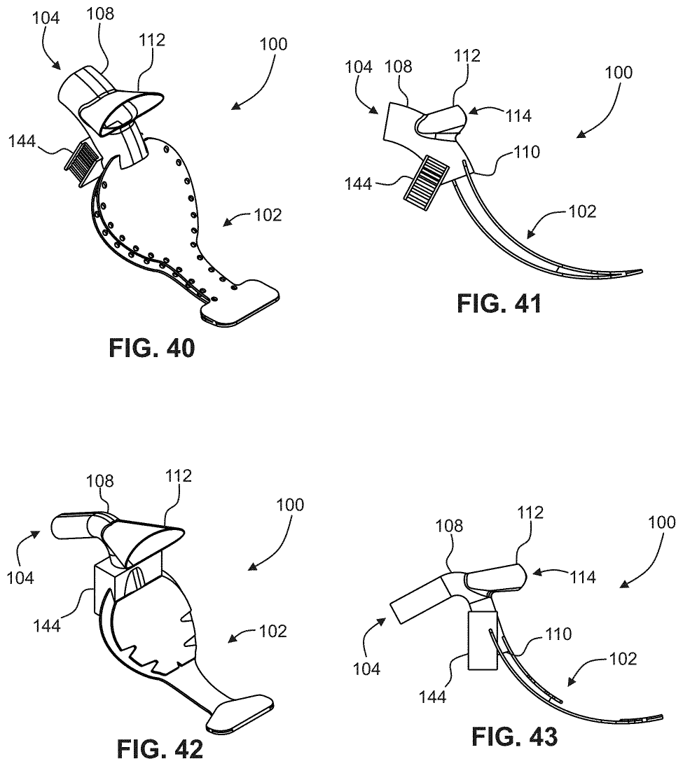

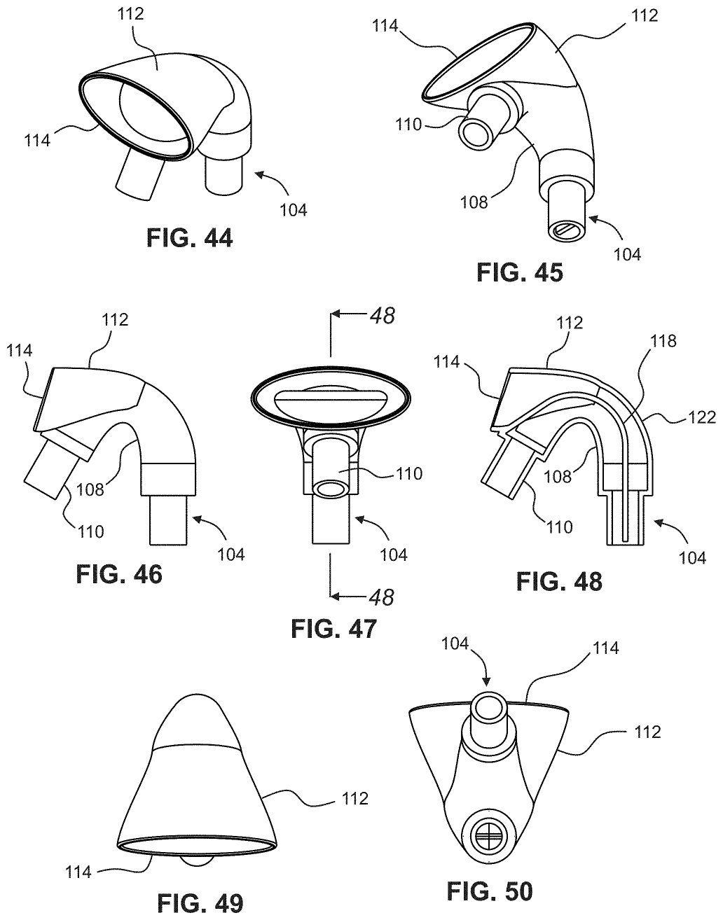

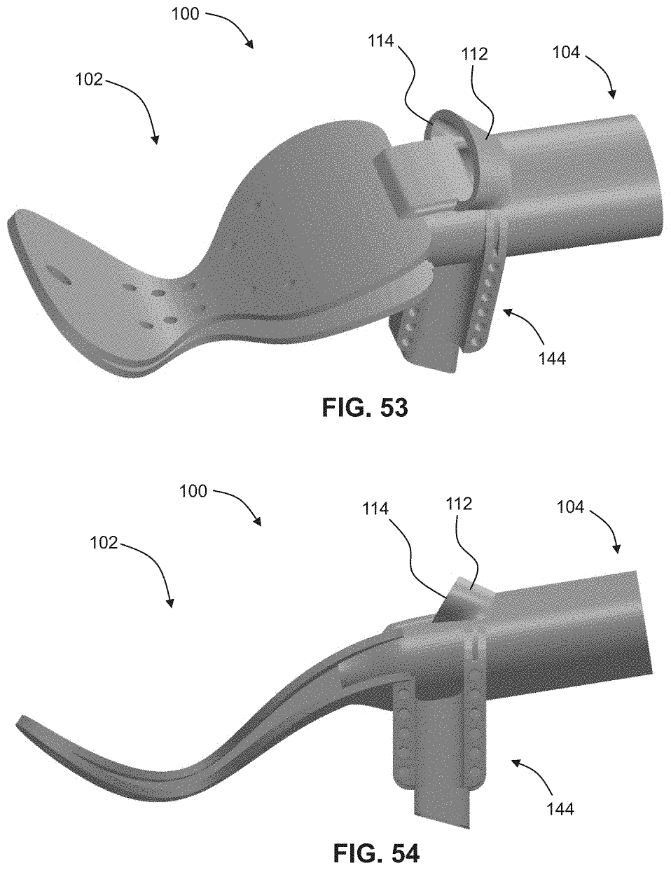

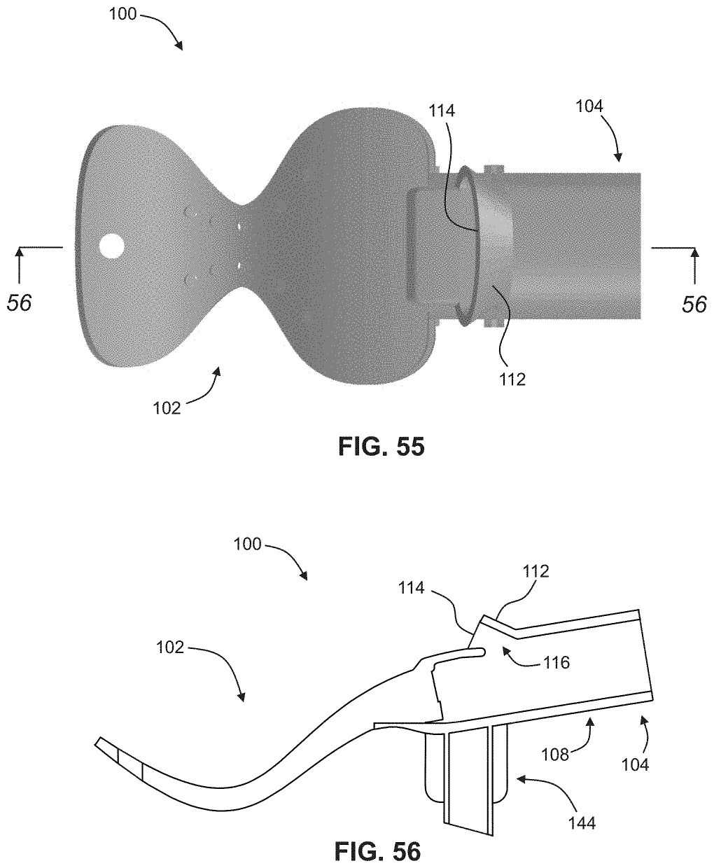

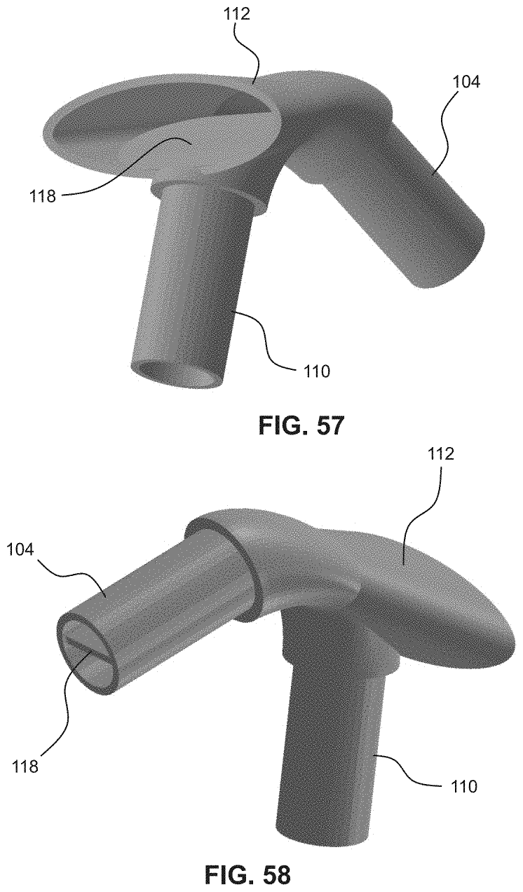

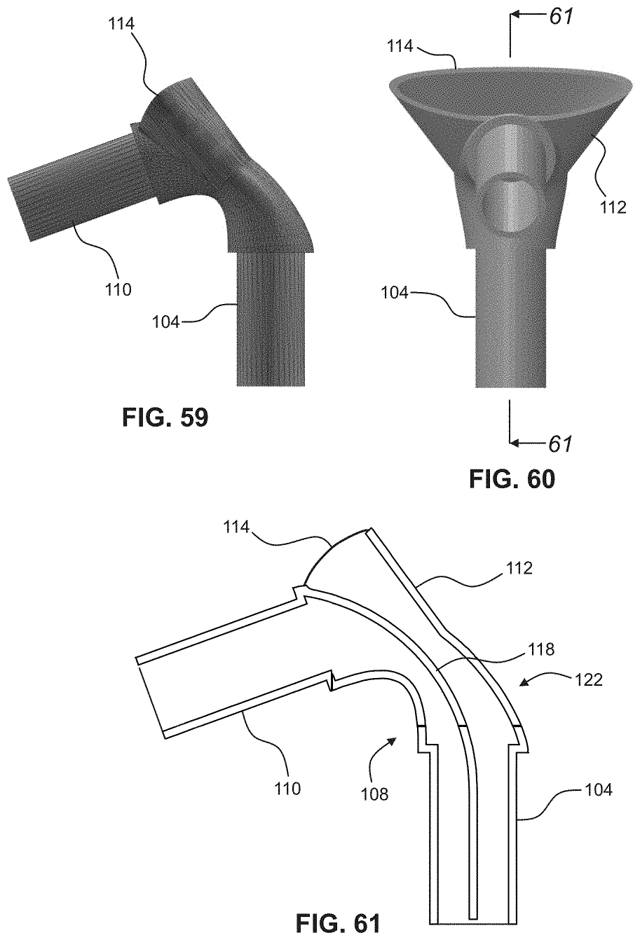

Certain examples of the dental suction device 100 may be configured to incorporate, as the oral partition element 102, conventional intra-oral suction mouthpieces such as the type used, for example, in conventional dental products such as the Ivory® ReLeaf®, Zyris® Isolite®, DryShield®, and Zirc® Mr. Thursty®. In the alternative, the oral partition element 102 may be a non-conventional intra-oral suction mouthpiece. Accordingly, the oral partition element 102 may also be referred to herein as a mouthpiece. Certain embodiments of the oral partition element 102 may include a bite block 114. See, for example, FIGS. 27-28 and 40-43, and 51-56.

Referring to FIGS. 3, 14 and 19, the first suction tube 108 may have a partition interface 110 attached (permanently or re-releasably) to the oral partition element 102, and may extend in fluid communication between the partition interface 110 and the suction interface portion 104. FIG. 32 illustrates an example dental suction device 100 in which the oral partition element 102 is permanently affixed to the first suction tube 108 by way of the partition interface 110 (for example, by way of co-molding the first suction tube 108 and the oral partition element 102, or by molding these features as a single unitary component using the same resin and injection). In the alternative, referring to FIG. 14, the oral partition element 102 may initially be formed as a separate component from the first suction tube 108, and then these two components may be permanently attached to one another by way of the partition interface 110.

The aerosol capture shroud 112 may have an aerosol inlet end 114 and an aerosol discharge end 116. The aerosol inlet end 114 may preferably be larger than the aerosol discharge end 116 by way of example, with reference to FIGS. 15 and 17, as the aerosol capture shroud 112 extends from the aerosol discharge end toward the aerosol inlet end 114, its cross-sectional area (orthogonal to the flow direction through shroud 112) may expand within plane 124, in an outward direction 126 of plane 124, or both. Moreover, the expansion of the aerosol capture shroud 112 along its length may follow a curved pathway, as illustrated for example if FIG. 15, similarly to a flared bell-shaped outlet of a musical wind instrument.

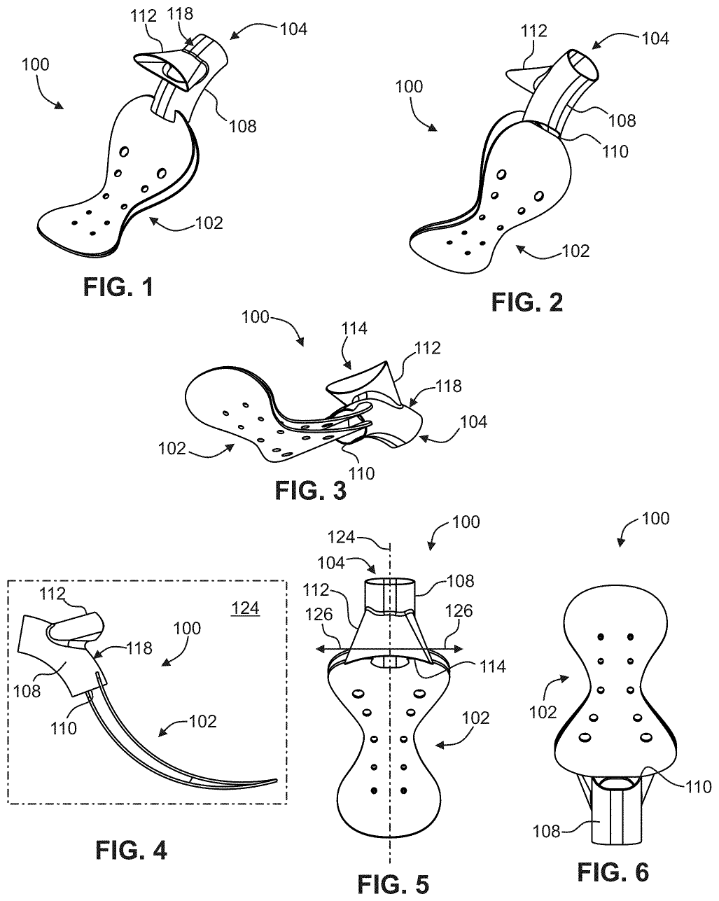

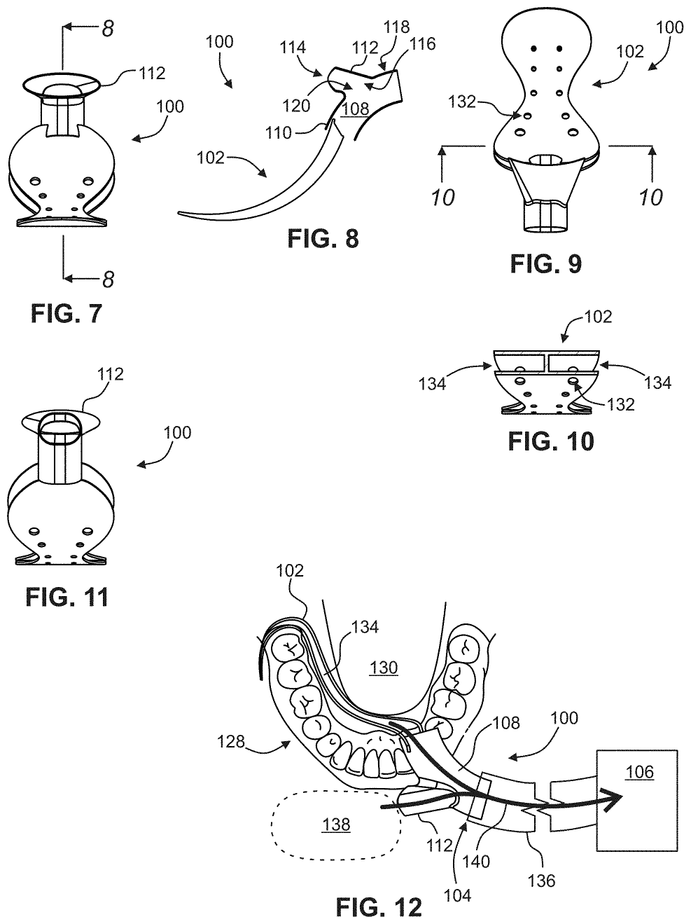

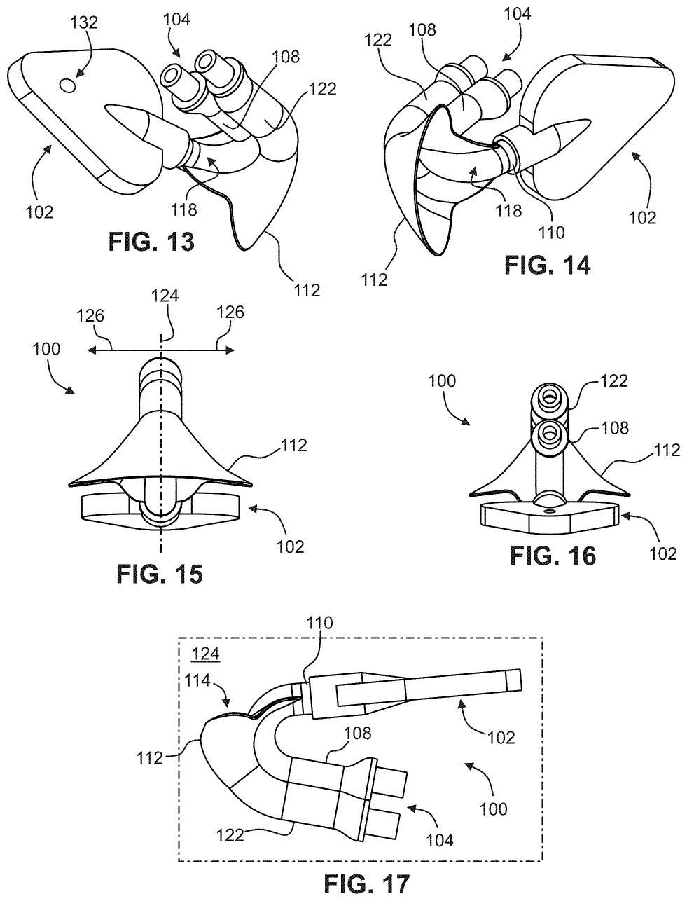

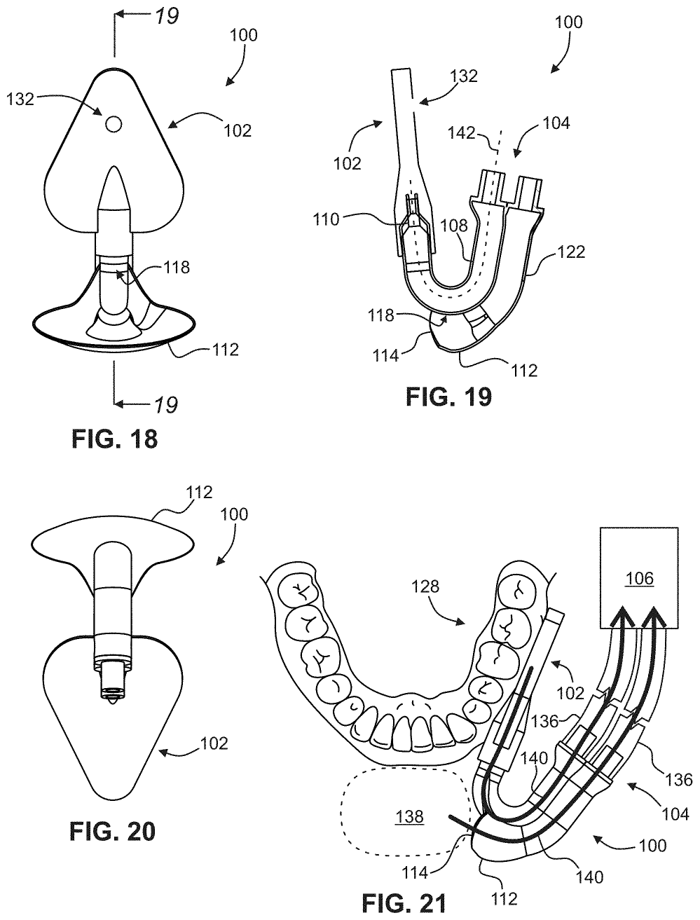

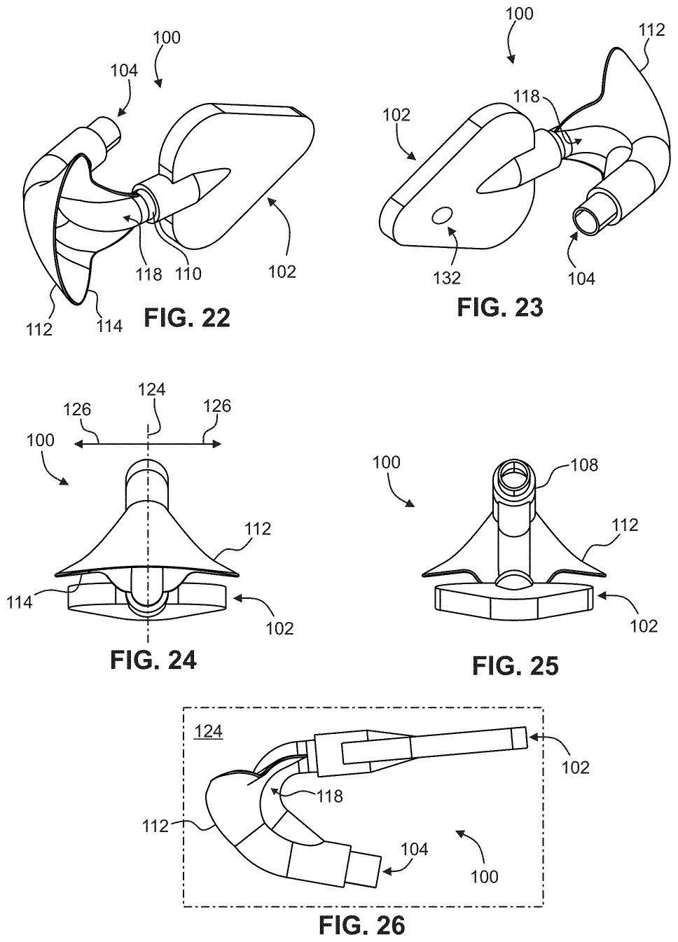

Referring to FIGS. 1 and 14, the aerosol capture shroud 112 may be affixed to an external surface 118 of the first suction tube 108. Moreover, referring to FIGS. 8 and 28, the first suction tube 108 may include an intermediate suction port 120, and the aerosol discharge end 116 may be in fluid communication with the first suction tube 108 by way of the intermediate suction port 120.

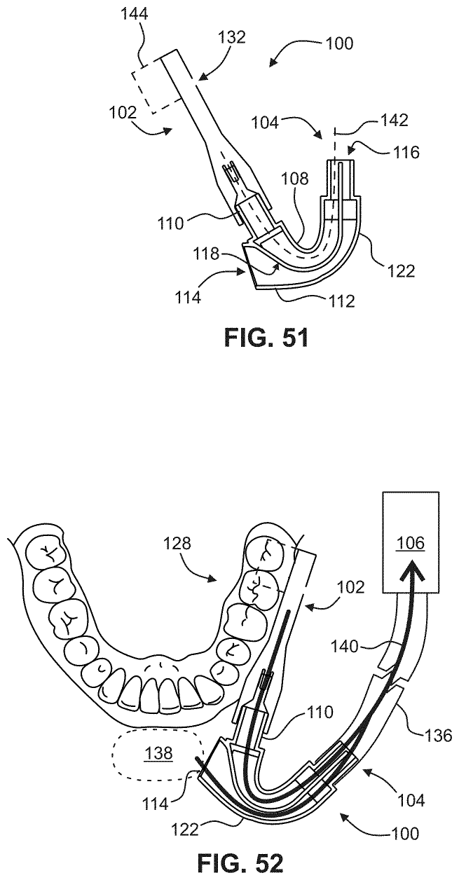

Referring to FIGS. 14, 32 and 51, particular examples of the dental suction device 100 may further comprise a second suction tube 122. The second suction tube may extend in fluid communication between the aerosol inlet end 114 and the suction interface portion 104. In certain such examples, the fluid communication between the aerosol discharge end 116 and the suction interface portion 104 may be entirely by way of the section suction tube 122 and may be independent of the first suction tube 108. For example, the first suction tube 108 and the second suction tube 122 may be formed of two independent lengths of tubing. In other examples of the dental suction device 100, the second suction tube 122 may extend in fluid communication from the aerosol inlet portion 144 to an intermediate suction port 120 of the first suction tube 108, or may partially comprise the outer surface 118 of the first suction tube 108 (see, for example, FIGS. 37 and 51).

Referring to FIGS. 19, 28 and 51, in particular examples of the dental suction device 100, the first suction tube 108 may be bent more than 90 degrees. For example, the first suction tube 108 may be curved along a tube curvature pathway 142 that is curved more than 90 degrees. In certain preferred embodiments, the first suction tube 108 may be bent more than 120 degrees. In examples of the dental suction device 100 having a first suction tube 108 and a second suction tube 122, the first suction tube 108 and section suction tube 122 may both be bent more than 90 degrees. Alternative or in addition, the second suction tube 122 may follow a portion of the curvature of the first suction tube 108 (as shown, for example, in FIG. 37).

Referring to FIGS. 12, 21, 30, 39 and 52, respective examples of the dental suction devices 100 are illustrated in use on a dental patient during a dental procedure. Generally, the oral partition element 102 is inserted and maintained within the oral cavity, either between a row of teeth 128 and the tongue 130 of the patient (as shown for example in FIG. 12), or between the row of teeth 128 and the cheek (not shown) of the patient (as shown for example in FIG. 21). The oral partition element 102 is typically comprised of a flexible polymer, so as to easily conform with the immediately adjacent features of the patient's mouth. During a typical dental procedure, excess saliva is suctioned from the oral cavity by way of the oral partition element 102. Referring to FIGS. 9 and 10, the oral partition element 102 may include suction apertures 132 and/or suction channels 134 therein for receiving the excess saliva. The excess saliva is generally suctioned from the oral partition element through the first suction tube 108. The excess saliva is then forced from the first suction tune 108 to the source of suction 106 by way of a suction conduct 136 connected in fluid communication therebetween. During a typical dental procedure, aerosol containing saliva, blood, debris and microorganisms may be expelled from the oral cavity and into the extraoral zone 138. The aerosol capture shroud 112 draws in this aerosol and directs it to the source of suction 106 by way of the first suction tube 108, a second suction tube 122, or a combination thereof. Depending upon the particular dental suction device 100, one or two respective suction conduits 136 may be removably affixed to the suction interface portion 104. Respective flow paths for the saliva and aerosol are illustrated at 140.

The following listing matches certain terminology used within this disclosure with corresponding reference numbers used in the non-limiting examples illustrated in the several figures.
100 dental suction device
102 oral partition element
104 suction interface portion
106 source of suction
108 first suction tube
110 partition interface
112 aerosol capture shroud
114 aerosol inlet end (of aerosol capture shroud)
116 aerosol discharge end (of aerosol capture shroud)
118 external surface (of first suction tube)
120 intermediate suction port (of first suction tube)
122 second suction tube
124 suction plane
126 outward directions (from suction plane)
128 row of teeth (of patient)
130 tongue (of patient)
132 suction apertures (of oral partition element)
134 suction channels (of oral partition element)
136 suction conduit
138 extraoral zone
140 flow path (e.g., of suctioned fluid and/or aerosol)
142 tube curvature pathway
144 bite block

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A hands-free dental suction device comprising:
an oral partition element;
a suction interface portion configured to be in fluid communication with a source of suction;
a first suction tube having a partition interface attached to the oral partition element, and extending in fluid communication between the partition interface and the suction interface portion; and
an aerosol capture shroud having an aerosol inlet end and an aerosol discharge end.

2. A hands-free dental suction device as defined in claim 1, wherein the aerosol inlet end is larger than the aerosol discharge end.

3. A hands-free dental suction device as defined in claim 2, wherein the aerosol capture shroud is affixed to an external surface of the first suction tube.

4. A hands-free dental suction device as defined in claim 3, wherein
the first suction tube includes an intermediate suction port; and
the aerosol discharge end is in fluid communication with the first suction tube by way of the intermediate suction port.

5. A hands-free dental suction device as defined in claim 3 further comprising a second suction tube, the second suction tube extending in fluid communication between the aerosol inlet end and the suction interface portion.

6. A hands-free dental suction device as defined in claim 5 wherein the fluid communication between the aerosol discharge end and the suction interface portion is independent of the first suction tube.

7. A hands-free dental suction device as defined in claim 5 wherein the second suction tube partially comprises the outer surface of the first suction tube.

8. A hands-free dental suction device as defined in claim 1 wherein the first suction tube is bent more than 90 degrees.

9. A hands-free dental suction device as defined in claim 5 wherein the first suction tube and second suction tube are bent more than 90 degrees.
